(19) United States
(12) Patent Application Publication
(10) Pub. No.: US 2004/0067528 A1
(43) Pub. Date: Apr. 8, 2004

(54) Method of detecting hydroxyproline and kit for detection

(76) Inventors: Katsu Honzawa, Hamamatsu-shi, Shizuoka (JP); Yukiko Sato, Hamamatsu-shi, Shizuoka (JP)

Correspondence Address: MORGAN LEWIS & BOCKIUS LLP, 1111 PENNSYLVANIA AVENUE NW, WASHINGTON, DC, 20004, US

(21) Appl. No.: 10/399,714

(22) PCT Filed: Oct. 24, 2001
(86) PCT No.: PCT/JP01/09330

(30) Foreign Application Priority Data
Oct. 24, 2000 (JP) 2000-32493

(51) Int. Cl. G01N 33/53; G01N 33/543

(52) U.S. Cl. 435/7.1; 436/518

(57) ABSTRACT

A method for detecting hydroxyprolines and a detection kit therefor are provided. A kit for detecting a hydroxyproline comprising a buffering agent or the like that is obtained from the immobilization of 4-chrolo-7-nitrobenzofurazane or 4-fluoro-7-nitrobenzofurazane on a solid phase is used to react with a sample containing a hydroxyproline; and the resultant reaction product is measured for its characteristic fluorescence (or absorption) to selectively detect the hydroxyproline with high sensitivity, rapidness and easiness.

### TECHNICAL FIELD

This invention generally relates to a method for detecting amino compounds and to a detection kit to be used in the method. More particularly, it relates to a method for detecting a specific amino compound (e.g., hydroxyproline) as well as to a detection kit to be used in the method.

### BACKGROUND ART

In various fields, it is considered necessary to detect amino compounds in a sample. Specifically, it is commonly practiced in the sites for food processing, such as food-processing factories, that equipment and machines for manufacturing foods are cleaned at every production lot. Examination of residues such as amino acids allows the degree of cleanness to be determined and the conditions of contamination to be confirmed. The detection and quantification of amino acids in a food are also conducted for the purposes of nutritional evaluation or food quality inspection.

Color reaction has been traditionally used to detect amino compounds, and ninhydrin is well known as a common coloration reagent for amino acids. Although the color reaction with ninhydrin is convenient, this reaction is positive for peptides or proteins other than amino acids and also causes coloration by amines, ammonia or urea derivatives. Then, coloration reagents to develop color for specific amino compounds are also known, which include Ehrlich's reagent (tryptophan) and Millon's reagent (tyrosine). These reagents usually detect the specific amino compounds if characteristic colors can be observed when they are added to provided solutions for testing (i.e., samples) and warmed or heated.

In addition, it has been carried out that a specific amino acid is detected in a sample (a biological sample) and it is utilized as a so-called marker for the diagnosis or the treatment of a disease. One example of such specific amino acids has been a hydroxyproline.

3-hydroxyproline, which is an isomer of the hydroxyprolines, attracts attention from the standpoint of collagen metabolism. Especially, it is recognized that the excretion of 3-hydroyproline into urine increases notably in association with malignant neoplasm, in metastases such as breast cancer, prostate cancer and lung cancer. This is because 3-hydroxyproline is an amino acid specific for collagen that constitutes the basement membrane enclosing a cell and it is excreted into urine once the cancer proliferates and the basement membrane is broken.

4-hydroxyproline, another isomer of the hydroxyprolines, can be used as a marker for bone resorption in determining bone metabolism; it is useful for the diagnosis of osteoporosis and for the selection of a therapeutic agent therefor as is the measurement of bone quantity by DPA, DEXA, QCT or the like.

Hydroxyprolines are thus important biochemical markers in the diagnosis of various malignant tumors and osteoporosis among others, and there is an urgent need to establish its convenient detection in biological samples.

For the methods of detection of 3-hydroxyproline that have been recognized in the past, there are known the direct measurement of its absorption spectrum and the method for measuring the absorption spectrum of a colored substance derived from 3-hydroxyproline through the ninhydrin reaction mentioned above or the like. However, the method does not determine 3-hydroxyproline selectively, but will allow regular constitutive amino acids to be detected concurrently.

Where other amino compounds (such as amino acids) coexist with 3-hydroxyproline in the sample, it will be necessary to separate 3-hydroxyproline in advance by suitable means. Such separation means requires equipment including high performance liquid chromatogram and it is difficult to selectively detect 3-hydroxyproline in the sample with rapidness and easiness, which has been a problem. In addition, because high performance chromatography is time-consuming in separation and washing of the column, the method finds difficulty in processing a large number of specimens at one time; and it experiences problematic contamination and reproducibility since different samples pass through the same column.

Accordingly, there is a strong need for a method for selectively detecting (including quantifying) 3-hydroxyproline or 4-hydroxyproline (which may be collectively referred to as “hydroxyproline(s)” in the present specification) in a sample solution without carrying out pretreatment (such as separation) and with rapidness and easiness.

### DISCLOSURE OF THE INVENTION

An object of this invention is to provide a method for the accurate and convenient detection of a specific amino compound and a kit for such detection. Further, another object of the invention is to provide a method capable of conducting simultaneous detection of the specific amino compound with accuracy and easiness even where a large number of sample specimens are present as well as to provide a kit for detection to be used in said method.

More concretely, this invention aims at providing a method for selectively detecting only a hydroxyproline (3-hydroxyproline or 4-hydroxyproline) in a sample with high sensitivity, rapidness and easiness, as well as a kit for such detection. Especially, the invention aims at providing a method and a kit capable of such detection even where a large number of sample specimens are present.

As a result of having repeatedly conducted diligent and intensive research to accomplish the above objects in view of the various problems in the prior art, the present inventors discovered that when a sample containing a hydroxyproline is added to 4-chloro-7-nitrobenzofurazan (hereafter referred to as “NBD-Cl”) or 4-fluoro-7-nitrobenzofurazan (hereafter referred to as “NBD-F”) immobilized on a solid phase and is allowed to react with it, only the hydroxyproline is led to a reaction product and the fluorescence spectrum or absorption spectrum measurement of this product permits the presence of the hydroxyproline to be determined and the content of the hydroxyproline to be judged, if desired, from which the completion of this invention resulted.

That is, this invention provides a method for detecting an amino compound in a sample by utilizing a detection reagent capable of specifically detecting the amino compound, characterized in that the detection reagent is immobilized on a solid phase.

The invention also provides the method for detection as described above, wherein the amino compound is a hydroxyproline and the detection reagent is 4-chloro-7-nitrobenzofurazan or 4-fluoro-7-nitrobenzofurazan.

The invention also provides the method of detection as described above, wherein the specific detection is conducted by measuring the fluorescence or the absorption of a reaction product between the hydroxyproline and 4-chloro-7-nitrobenzofurazan or 4-fluoro-7-nitrobenzofurazan.

The invention also provides a kit for detecting an amino compound comprising at least, a detection reagent immobilized on a solid phase capable of specifically detecting the amino compound and a buffering agent.

Further, the invention provides a method for detecting a hydroxyproline in a sample, the method comprising conducting at least the following steps of:

a) immobilizing a detection reagent of 4-chloro-7-nitrobenzofurazan onto a solid phase:

b) allowing the hydroxyproline to react with 4-chloro-7-nitrobenzofurazan immobilized on the solid phase: and

c) measuring the fluorescence or the absorption of a reaction product formed in step b).

The invention also provides the method for detecting a hydroxyproline in a sample as described above, wherein the immobilization in step a) is conducted in the presence of a surfactant and/or a dispersing adjuvant.

The invention also provides the method for detecting a hydroxyproline in a sample as described above, wherein in step b) the reaction is carried out for only a sufficient time during which merely the reaction between the hydroxyproline and 4-chloro-7-nitrobenzofurazan are reacted when any amino compound other than the hydroxyproline is present in the sample.

In addition, the invention provides a kit for detecting a hydroxyproline comprising at least, 4-chloro-7-nitrobenzofurazan immobilized on a solid phase, a buffering agent, and an acid solution.

### BRIEF DESCRIPTION OF THE DRAWINGS

FIG. 1 is a representation showing a reaction scheme between 3-hydroxyproline and NBD-Cl on which the detection method of this invention is based, as well as a deduced structure of the reaction product therefrom.

FIG. 2 is a figure showing a spectrum as measured by fluorescence where a mixed sample containing 3-hydroxyproline, other 20 different species of amino acids and a control sample is treated with a detection kit of the invention (fluorescence spectrum resulting from reaction with an amino acid mixture).

### BEST MODE FOR CARRYING OUT THE INVENTION

According to this invention, there is provided a method for detecting an amino compound in a sample by utilizing a detection reagent capable of specifically detecting the amino compound, characterized in that the detection reagent is immobilized on a solid phase.

According to the invention, there is also provided a kit for detecting an amino compound comprising at least, a detection reagent capable of specifically detecting the amino compound immobilized on a solid phase and a buffering agent, which kit is suited to practicing the detection method described above.

The amino compounds that may be detected by the detection method and the detection kit according to the invention are not particularly limited insofar as they are detectable amino compounds when combined with the detection reagent and, for example, include glycine, alanine, valin, serine, leucine, glutamic acid, proline, and hydroxyprolines: as used in the present specification, “detection or detect” imply both determination of the presence or the absence and quantification and is used equivalently to “assay.” This invention will be described in detail by reference to embodiments thereof, where hydroxyprolines are taken as concrete examples.

[Hydroxyproline (s)]

The hydroxyprolines that can be detected in accordance with the invention encompass isomers with respect to the position of the hydroxyl group. They are 3-hydroxyproline, 4-hydroxyproline and 5-hydroxyproline; 3-hydroxyproline and 4-hydroxyproline, which are biochemical markers for certain diseases, are important as the target to be detected. Among these, of particular importance is 3-hydroxyproline, a tumor marker. Two asymmetric carbon atoms exist for each of the hydroxyprolines. The hydroxyprolines according to the invention, therefore, embrace all the possible optical isomers as well as any mixtures of the isomers. Further, the hydroxyprolines according to the invention usually mean L-forms, but the detection method or the detection kit according to the invention is also applicable to the detection of hydroxyprolines in D-forms.

[Samples]

The sample containing a hydroxyproline that is subjected to the detection method or the detection kit according to the invention (which may be referred to as “the sample according to the invention” hereafter) is in a solution state and it is not particularly limited to this property. There can be used an aqueous solution, a solution in organic solvent, and a mixed solution of water and organic solvent. Here, the appropriate solvent is the one that is miscible with water. Specifically, dimethylsulfoxide (DMSO), dimethylformamide (DMF), ethanol and the like are mentioned. When the sample is a biological sample, the sample preparation in an aqueous solution is especially preferred.

The concentration of a hydroxyproline in the sample according to the invention is also not particularly limited and the concentration suited to the measurement of fluorescence or absorption spectrum may suffice. When the concentration is too high in the measurement, it can be adjusted to an optimal concentration by dilution with an appropriate solvent (which may contain a buffer and water). Further, when the concentration in the sample is too low for the spectrum measuring conditions, it can be concentrated to an appropriate concentration. Specifically, the means for concentration include removal of solvent, chromatography, etc.

The sample according to this invention is presumed or suspected to contain a hydroxyproline, particularly in the case of a biological sample. It is thus possible that the sample does not substantially contain the hydroxyproline as a result of having practiced the detection method of the invention: that is, the hydroxyproline is not detected.

When any fluorescent substance is coexistent in the sample according to the invention, the fluorescence of the fluorescent product resulting from a hydroxyproline and the fluorescence of the fluorescent substance can possibly overlap. In such a case, it is preferred that pretreatment be carried out to remove the fluorescent substance having coexisted in the sample. Concretely, there are mentioned a method for removing the fluorescent substance by extraction with a suitable solvent, a method for removing the fluorescent substance by absorption with a suitable absorbent, etc. Alternatively, where the pretreatment is not to be conducted, there are mentioned a method for waveform-separating from the overlapped fluorescence spectrum, the spectrum due to a hydroxyproline, a method for measuring only the spectrum due to a hydroxyproline by selecting the excitation wavelength, etc. When the detection method of this invention employs absorption spectra in place of fluorescence spectra, the same is true.

When two or more isomers of the hydroxyprolines (e.g., 3-hydroxyproline and 4-hydroxyproline) are coexistent in a sample according to the invention, there is a possibility that these isomers cannot be differentiated in the measurement of fluorescence or absorption. In these instances, the objective hydroxyproline is separated by a separation means such as high performance chromatography beforehand and the detection method of the invention may be carried out.

It will be possible to measure a hydroxyproline in a biological sample containing urine or a mixture of proteins by combining any of the treatment methods described above.

(Detection Reagents)

The detection method and the detection kit according to this invention employ NBD-Cl or NBD-F. It is known in the art that these compounds may be used for the fluorometry of amino compounds or amino acids. For example, JPA 7-313179 discloses a method of producing 4-hydroxyproline by enzyme-catalyzed hydroxylation of L-prolines. Here, after the reaction product containing prolines is separated by high performance liquid chromatography, the resulting 4-hydroxyproline is detected with NBD-Cl. The use of NBF-F in the fluorescence detection and quantification is also disclosed in JPA 63-22261. However, it was not apparent from these prior art references that hydroxyprolines in a mixture of amino compounds (e.g., amino acids) or proteins could selectively be detected using NBD-Cl or NBD-F. Not to mention, there is neither disclosure nor suggestion as to the use of immobilized NBD-Cl or immobilized NBD-F in the detection of any amino compound: it has thus been totally unexpected.

NBD-Cl and NBD-F are preferable detection reagents when hydroxyprolines are targeted. Given a specific amino compound other than hydroxyprolines, the detection reagents capable of detecting it are often known by one skilled in the art. Preferably, the detection reagents are those labeled with chromophores, fluorophores, phosphorophores, chemiluminiscent groups or the like.

[Solid Phase]

This invention is characterized in that the detection reagent for an amino compound exemplified by NBD-Cl (or NBD-F) as described above is immobilized on a solid phase. There can be used tubes (made of glass or plastic), including microtubes, plates (such as 96/384), pipette tips, polystyrene beads, latex particles, magnetic particles, etc; and their materials or shapes are not limited at all. The immobilization method may utilize physical adsorption, covalent bonding and other techniques known in the art. When the detection reagent is immobilized on the solid phase by physical adsorption, a dispersing adjuvant and/or a surfactant may be used to immobilize the reagent with uniform scattering. Suitable dispersing adjuvants include polyethylene glycol, cyclodextrin, dextran, etc. Suitable surfactants include sodium dodecyl sulfate (SDS), Triton-X, Tween-20, etc.

[Reaction Conditions]

According to a preferred embodiment of this invention, the method for detecting a hydroxyproline is characterized by conducting at least the following steps of:

a) immobilizing a detection reagent of NBD-Cl onto a solid phase;

b) allowing the hydroxyproline to react with NBD-Cl immobilized on the solid phase; and

c) measuring the fluorescence or the absorption of a reaction product formed in Step b).

Step a) comprises immobilizing NBD-Cl onto a solid phase as stated above. Step b) comprises adding a sample (containing a hydroxyproline) to the immobilized NBD-Cl and allowing NBD-Cl to react with the hydroxyproline in the sample.

For the preparation of a sample containing a hydroxyproline, a suitable solvent is used to prepare the sample with an appropriate concentration, as described previously. In so doing, a buffer is preferably used to maintain a constant pH of the reaction system (desirably, a PH in the range of from about 4.0 to about 8.5). Specifically, there are mentioned a borate buffer, phosphate buffer and Tris buffer. Alternatively, the detection kit may be provided with a buffering agent. The buffering agent may be either solid or liquid (i.e., buffer (solution)); and in case the former is employed, it takes the forms of granule, powder or tablet, among others, and is dissolved into purified water or the like to prepare a buffer at the time of use.

Under the conditions set as described above, the reaction proceeds by incubation at room temperature (about 20 to about 50° C.) for a predetermined time. Preferably, reaction is allowed for only a sufficient time during which merely the hydroxyproline and NBD-Cl (or NBD-F) are reacted. This is because other amino compounds (e.g., amino acids), which may be present in the sample, has low reactivity to NBD-Cl under the set reaction conditions and there is almost no formation of reaction products resulting from the other amino compounds when the reaction is quenched at the point reaction between the hydroxyproline and NBD-Cl has substantially ended. For this reason, the reaction time is preferably set at between about one and about two minutes.

Step c) comprises the step of measuring the fluorescence or the absorption of the reaction product formed in Step b).

Immediately after Step b), the reaction system may be subjected to fluorescence or absorption measurement, but the measurement should preferably be done after the reaction system has been acidified in order to have the detection sensitivity enhanced, in accordance the detection method of this invention. For the acids used to this end, there can be used inorganic acids and organic acids. Specifically, mineral acids such as hydrochloric acid and sulfuric acid are preferable.

(Reaction Products)

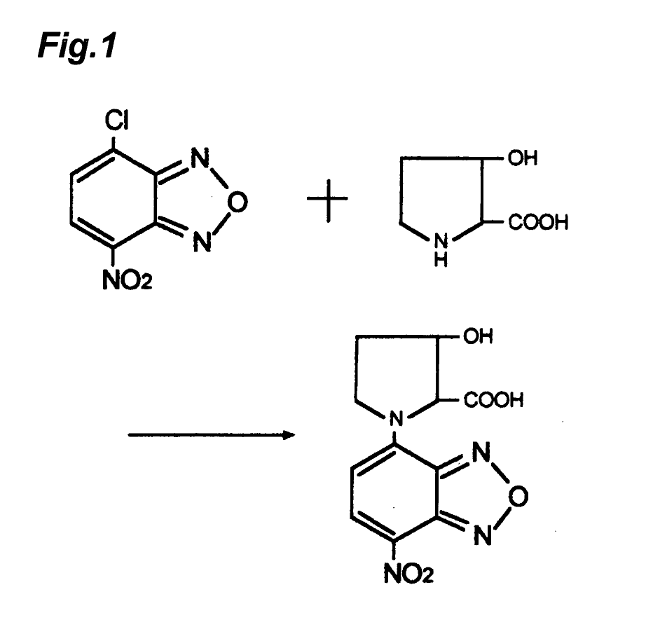

Regardless of whether the reaction system is neutralized or not, there is formed a reaction product between the hydroxyproline and NBD-Cl (or NBD-F) in the obtained reaction solution. The reaction product is only resulted from the hydroxyproline and it is thought that the hydroxyproline reacts with NBD-Cl approximately at 1:1. Therefore, when the hydroxyproline used (or present) is 3-hydroxyproline and the detection reagent is NBD-Cl, the reaction product is presumed to have a structure shown in FIG. 1. However, the correctness of the deductive structure does not affect practicing this invention in any way.

(Fluorescence or Absorption Measurement)

The reaction product has an emission (fluorescence) wavelength at about 550 nm when it is derived from 3-hydroxyproline (and also from NBD-Cl). It is also provided with an absorption peak at about 500 nm. Devices or methods capable of measuring such fluorescence or absorption can therefore be used in this invention without any particular limitations. Concretely, any device capable of measuring absorption spectra in the visible area in the range of from about 350 to about 600 nm is adequate. Any device capable of using a fluorescence excitation wavelength of from about 250 to about 550 nm and of measuring fluorescence spectra in the range of from about 350 to about 600 nm is adequate.

When the characteristic spectrum (or absorption spectrum) is observed at a significant level, it is determined that the hydroxyproline is present in the sample. In addition, it is possible to prepare a calibration curve of concentration vs. fluorescence intensity or concentration vs. absorption intensity based on the known hydroxyproline in advance. The calibration curve may be used to quantify the hydroxyproline in the sample. This quantification method is useful where a biological sample such as urine is subjected to detection according to this invention; and the content of the hydroxyproline may be calculated in urine.

Although two types of measuring methods, fluorescence measurement or absorption measurement, are possible in the detection method of this invention, the fluorescence measurement is more preferable from the standpoint of detection sensitivity in the case of the biological sample containing a very minute amount of hydroxyproline.

(Detection Kit)

A kit of this invention for detecting an amino compound is characterized by comprising at least, a detection reagent immobilized on a solid phase capable of specifically detecting the amino compound and a buffering agent. The kit of the invention for detecting a hydroxyproline, which constitutes a preferred embodiment, comprises at least, 4-chloro-7-nitrobenzofurazan immobilized on a solid phase, a buffering agent, and an acid solution (preferably, hydrochloric acid solution). It refers to that in which those are provided in a complete set at the time of detection, concretely that which is disclosed in the Examples. The detection kit may further contain a specimen-diluted solution, a dissolution adjuvant, a preservative, a stabilizer, a disintegrating agent, and other additives if necessary. In addition, when the quantification of the amino compound is desired, it is preferred that the kit contain a normal concentration solution of its standard compound.

This invention will be described in detail by way of examples; however, the invention should not be limited by these examples.

### EXAMPLES

1. Preparation of Immobilized NBD-Cl

To each well of a 96-well plate was added an acetonitrile solution (100 μl) of 10 mM NBD-Cl containing 0.01% Triton-X and 0.5% polyethylene glycol and the plate was dried in an oven at 30° C. for 2 hours. The solid phase (96-well plate) having this immobilized NBD-Cl can be stored in a cool dark place once sealed under a nitrogen atmosphere.

2. Preparation of Sample Solutions

Various types of amino acids were dissolved in a borate buffer (pH 6.8) to prepare a mixed sample solution such that each amino acid is 1 mM. The amino acids used are, in addition to 3-hydroxyproline, alanine, arginine, asparagine, asparagic acid, cysteine (hydrochloride), glutamic acid, glutamine, glycine, histidine, isoleucine, leucine, lysine (hydrochloride), methionine, phenylalanine, proline, serine, threonine, tryptophan, tyrosine and valine, which totaled 21 species. These amino acids were all L-forms.

3. Preparation of Control Sample Solution

Except for 3-hydroxyproline, the used amino acids were employed to prepare a mixed sample solution of 20 different species of amino acids (each amino acid 1 mM, borate buffer (pH 6.8)).

4. Detection Reaction and Fluorescence Spectrum Measurement

The sample solution (100 μl) prepared in 2. above was added to the 96-well plate immobilized with NBD-Cl according to 1, and reaction was allowed at room temperature for one minute, to which 100 μl of 100 mM HCl was then added.

In a similar manner, the control sample solution (100 μl) prepared in 3. above was added to the 96-well plate immobilized with NBD-Cl according to 1, and reaction was allowed at room temperature for one minute, to which 100 μl of 0.5 M HCl was then added.

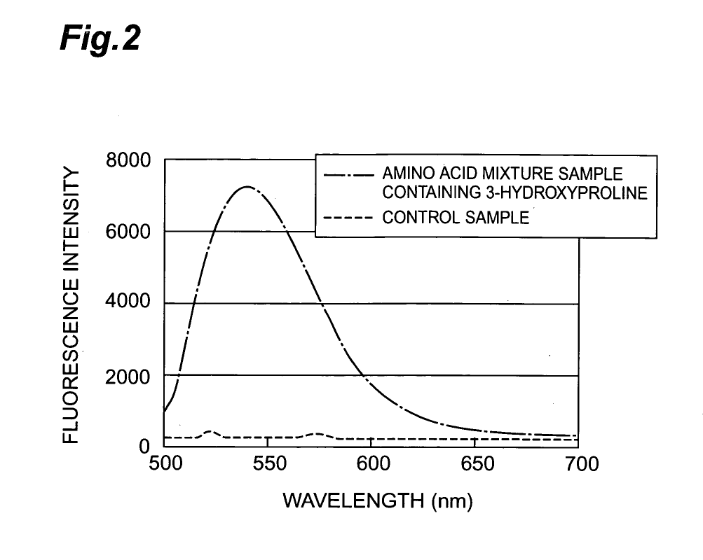

Among reaction solutions (sample and control) each 96-well plate was set on a fluorescence plate reader (Tekan Group Ltd: FLUOSTAR PRO) and fluorescence spectra were measured at an excitation wavelength of 505 nm. The measurement results are shown in FIG. 2. The fluorescence spectrum obtained from the sample solution containing 3-hydroxyproline greatly differs from the fluorescence spectrum from the control sample solution containing only a mixture of the other amino acids. Specifically in the sample containing 3-hydroxyproline, strong fluorescence was observed at approximately 550 nm post-reaction. This indicates that the reaction product between 3-hydroxyproline and NBD-Cl has resulted which posses said fluorescence.

In summary, the results from the fluorescence spectrum measurement have revealed that even in a sample where 20 species of different amino acids are coexistent with 3-hydroxyproline further at equal concentrations, the detection method of this invention can detect only 3-hydroxyproline. Consequently, it has been shown that 3-hydroxyproline can selectively be detected in such a mixture of amino acids without any manipulation for its separation.

### INDUSTRIAL APPLICABILITY

According to a detection method and a detection kit of this invention, a detection reagent immobilized on a solid phase capable of detecting an amino compound is used to detect the amino compound in a sample; therefore, the step of pretreatment of the sample can be omitted and a large number of sample specimens can be examined rapidly. The operation method of detection is also very convenient.

According to a detection method and a detection kit of this invention, in a sample containing a hydroxyproline a detection kit such that a detection reagent, NBD-Cl or NBD-F (preferably NBD-Cl), has been immobilized on a solid phase is used to measure the fluorescence or the absorption of the reaction product formed as a result of allowing the hydroxyproline to react with NBD-Cl or NBD-F; therefore, no manipulation for separation is necessary and it will be possible to detect only the hydroxyproline with high sensitivity, rapidness and easiness. In addition, it will be possible to examine a large number of sample specimens at one time.

Furthermore, according to a specific embodiment of the detection method and the detection kit of this invention, a detection reagent, NBD-Cl or NBD-F (preferably NBD-Cl), is immobilized on a solid phase, a sample containing a hydroxyproline and other amino compounds (e.g., amino acids) is allowed to react therewith for only a sufficient time during which merely the hydroxyproline and NBD-Cl are reacted, and the fluorescence or the absorption of the thus-obtained reaction product is measured; therefore, no manipulation for separation is necessary and it will be possible to selectively detect the hydroxyproline with high sensitivity, rapidness and easiness. In addition, it will be possible to examine a large number of sample specimens at one time.

1. A method for detecting an amino compound in a sample by utilizing a detection reagent capable of specifically detecting the amino compound, characterized in that the detection reagent is immobilized on a solid phase.

2. The method of detection according to claim 1, wherein the amino compound is a hydroxyproline and the detection reagent is 4-chloro-7-nitrobenzofurazan or 4-fluoro-7-nitrobenzofurazan.

3. The method of detection according to claim 2, wherein the specific detection is conducted by measuring the fluorescence or the absorption of a reaction product of the hydroxyproline with 4-chloro-7-nitrobenzofurazan or with 4-fluoro-7-nitrobenzofurazan.

4. A kit for detecting an amino compound comprising at least, a detection reagent immobilized on a solid phase capable of specifically detecting the amino compound and a buffering agent.

5. A method for detecting a hydroxyproline in a sample, the method comprising conducting at least the following steps of:
a) immobilizing a detection reagent of 4-chloro-7-nitrobenzofurazan onto a solid phase;
b) allowing the hydroxyproline to react with 4-chloro-7-nitrobenzofurazan immobilized on the solid phase; and
c) measuring the fluorescence or the absorption of a reaction product formed in step b).

6. The method for detecting a hydroxyproline in a sample according to claim 5, wherein the immobilization is conducted in the presence of a surfactant and/or a dispersing adjuvant.

7. The method for detecting a hydroxyproline in a sample according to claim 5, wherein in step b) the reaction is carried out for only a sufficient time during which merely the hydroxyproline and 4-chloro-7-nitrobenzofurazan are reacted when any amino compound other than the hydroxyproline is present in the sample.

8. A kit for detecting a hydroxyproline comprising at least, 4-chloro-7-nitrobenzofurazan immobilized on a solid phase, a buffering agent, and an acid solution.
